(19) United States
(12) Patent Application Publication
(10) Pub. No.: US 2022/0157425 A1
(43) Pub. Date: May 19, 2022

(54) FEEDBACK SYSTEM AND METHOD

(71) Applicant: Nuance Communications, Inc., Burlington, MA (US)

(72) Inventor: Raghu Vemula, Salem, NH (US)

(21) Appl. No.: 17/524,340

(22) Filed: Nov. 11, 2021

Related U.S. Application Data
(60) Provisional application No. 63/113,439, filed on Nov. 13, 2020.

(51) Int. Cl. G16H 15/00; G16H 30/40

(52) U.S. Cl. CPC G16H 15/00; G16H 30/40

(57) ABSTRACT

A computer-implemented method, computer program product and computing system for receiving a result set for content processed by an automated analysis process; receiving human feedback concerning the result set; and providing feedback information to the developer of the automated analysis process based, at least in part, upon the result set and the human feedback.

### RELATED APPLICATION

This application claims the benefit of U.S. Provisional Application No. 63/113,439, filed on 13 Nov. 2020, the entire contents of which is herein incorporated by reference.

### TECHNICAL FIELD

This disclosure relates to feedback systems and methods and, more particularly, to feedback systems and methods concerning artificial intelligence and machine learning functionality.

### BACKGROUND

Recent advances in the fields of artificial intelligence and machine learning are showing promising outcomes in the analysis of clinical content, examples of which may include medical imagery. Accordingly, processes and algorithms are constantly being developed that may aid in the processing and analysis of such medical imagery. Unfortunately, such processes and algorithms often need to be revised/finetuned to address inaccuracies and unanticipated results. Traditionally, when an unanticipated result occurs, the data that caused the unanticipated result is sent to the developer of the process/algorithm for trouble shooting.

### SUMMARY OF DISCLOSURE

In one implementation, a computer-implemented method is executed on a computing device and includes: receiving a result set for content processed by an automated analysis process; receiving human feedback concerning the result set; and providing feedback information to the developer of the automated analysis process based, at least in part, upon the result set and the human feedback.

One or more of the following features ay be included. The result set may be an auto-populated report. The human feedback may include amendments to the auto-populated report. The human feedback may concern the accuracy of the result set. The human feedback may include amendments to the result set. The content may be medical imagery. Providing feedback information to the developer of the automated analysis process may include: providing at least a portion of the result set and/or the human feedback to the developer of the automated analysis process. Providing feedback information to the developer of the automated analysis process may include: providing differential information that defines differences between the result set and the human feedback to the developer of the automated analysis process. The feedback information may be processed to remove confidential data. The feedback information may be processed to remove confidential data in accordance with one or more medical data privacy rules.

In another implementation, a computer program product resides on a computer readable medium and has a plurality of instructions stored on it. When executed by a processor, the instructions cause the processor to perform operations including: receiving a result set for content processed by an automated analysis process; receiving human feedback concerning the result set; and providing feedback information to the developer of the automated analysis process based, at least in part, upon the result set and the human feedback.

One or more of the following features ay be included. The result set may be an auto-populated report. The human feedback may include amendments to the auto-populated report. The human feedback may concern the accuracy of the result set. The human feedback may include amendments to the result set. The content may be medical imagery. Providing feedback information to the developer of the automated analysis process may include: providing at least a portion of the result set and/or the human feedback to the developer of the automated analysis process. Providing feedback information to the developer of the automated analysis process may include: providing differential information that defines differences between the result set and the human feedback to the developer of the automated analysis process. The feedback information may be processed to remove confidential data. The feedback information may be processed to remove confidential data in accordance with one or more medical data privacy rules.

In another implementation, a computing system includes a processor and a memory system configured to perform operations including: receiving a result set for content processed by an automated analysis process; receiving human feedback concerning the result set; and providing feedback information to the developer of the automated analysis process based, at least in part, upon the result set and the human feedback.

One or more of the following features ay be included. The result set may be an auto-populated report. The human feedback may include amendments to the auto-populated report. The human feedback may concern the accuracy of the result set. The human feedback may include amendments to the result set. The content may be medical imagery. Providing feedback information to the developer of the automated analysis process may include: providing at least a portion of the result set and/or the human feedback to the developer of the automated analysis process. Providing feedback information to the developer of the automated analysis process may include: providing differential information that defines differences between the result set and the human feedback to the developer of the automated analysis process. The feedback information may be processed to remove confidential data. The feedback information may be processed to remove confidential data in accordance with one or more medical data privacy rules.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a distributed computing network including a computing device that executes an online platform process according to an embodiment of the present disclosure;

FIG. 2 is a diagrammatic view of medical data before and after processing;

FIG. 3 is a flowchart of the online platform process of FIG. 1 according to an embodiment of the present disclosure; and

FIG. 4 is a diagrammatic view of confidential data and related non-confidential data.

Like reference symbols in the various drawings indicate like elements.

### DETAILED DESCRIPTION OF THE PREFERRED EMBODIMENTS

System Overview

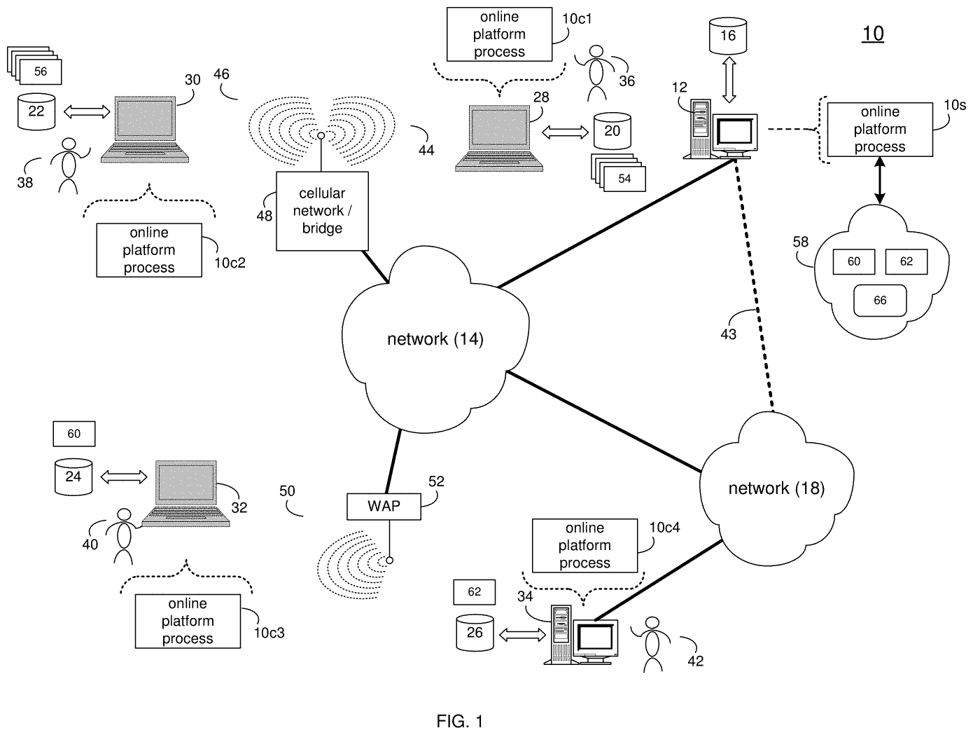

Referring to FIG. 1, there is shown online platform process 10. Online platform process 10 may be implemented as a server-side process, a client-side process, or a hybrid server-side/client-side process. For example, online platform process 10 may be implemented as a purely server-side process via online platform process 10s. Alternatively, online platform process 10 may be implemented as a purely client-side process via one or more of online platform process 10c1, online platform process 10c2, online platform process 10c3, and online platform process 10c4. Alternatively still, online platform process 10 may be implemented as a hybrid server-side/client-side process via online platform process 10s in combination with one or more of online platform process 10c1, online platform process 10c2, online platform process 10c3, and online platform process 10c4. Accordingly, online platform process 10 as used in this disclosure may include any combination of online platform process 10s, online platform process 10c1, online platform process 10c2, online platform process 10c3, and online platform process 10c4. Examples of online platform process 10 may include but are not limited to all or a portion of the PowerShare™ platform and/or the PowerScribe™ platform available from Nuance Communications™ of Burlington, Mass.

Online platform process 10s may be a server application and may reside on and may be executed by computing device 12, which may be connected to network 14 (e.g., the Internet or a local area network). Examples of computing device 12 may include, but are not limited to: a personal computer, a server computer, a series of server computers, a mini computer, a mainframe computer, or a cloud-based computing platform.

The instruction sets and subroutines of online platform process 10s, which may be stored on storage device 16 coupled to computing device 12, may be executed by one or more processors (not shown) and one or more memory architectures (not shown) included within computing device 12. Examples of storage device 16 may include but are not limited to: a hard disk drive; a RAID device; a random access memory (RAM); a read-only memory (ROM); and all forms of flash memory storage devices.

Network 14 may be connected to one or more secondary networks (e.g., network 18), examples of which may include but are not limited to: a local area network; a wide area network; or an intranet, for example.

Examples of online platform processes 10c1, 10c2, 10c3, 10c4 may include but are not limited to a web browser, a game console user interface, a mobile device user interface, or a specialized application (e.g., an application running on e.g., the Android™ platform, the iOS™ platform, the Windows™ platform, the Linux™ platform or the UNIX™ platform). The instruction sets and subroutines of online platform processes 10c1, 10c2, 10c3, 10c4, which may be stored on storage devices 20, 22, 24, 26 (respectively) coupled to client electronic devices 28, 30, 32, 34 (respectively), may be executed by one or more processors (not shown) and one or more memory architectures (not shown) incorporated into client electronic devices 28, 30, 32, 34 (respectively). Examples of storage devices 20, 22, 24, 26 may include but are not limited to: hard disk drives; RAID devices; random access memories (RAM); read-only memories (ROM), and all forms of flash memory storage devices.

Examples of client electronic devices 28, 30, 32, 34 may include, but are not limited to, a smartphone (not shown), a personal digital assistant (not shown), a tablet computer (not shown), laptop computers 28, 30, 32, personal computer 34, a notebook computer (not shown), a server computer (not shown), a gaming console (not shown), and a dedicated network device (not shown). Client electronic devices 28, 30, 32, 34 may each execute an operating system, examples of which may include but are not limited to Microsoft Windows™, Android™, iOS™, Linux™, or a custom operating system.

Users 36, 38, 40, 42 may access online platform process 10 directly through network 14 or through secondary network 18. Further, online platform process 10 may be connected to network 14 through secondary network 18, as illustrated with link line 43.

The various client electronic devices (e.g., client electronic devices 28, 30, 32, 34) may be directly or indirectly coupled to network 14 (or network 18). For example, laptop computer 28 and laptop computer 30 are shown wirelessly coupled to network 14 via wireless communication channels 44, 46 (respectively) established between laptop computers 28, 30 (respectively) and cellular network/bridge 48, which is shown directly coupled to network 14. Further, laptop computer 32 is shown wirelessly coupled to network 14 via wireless communication channel 50 established between laptop computer 32 and wireless access point (i.e., WAP) 52, which is shown directly coupled to network 14. Additionally, personal computer 34 is shown directly coupled to network 18 via a hardwired network connection.

WAP 52 may be, for example, an IEEE 802.11a, 802.11b, 802.11g, 802.11n, Wi-Fi, and/or Bluetooth device that is capable of establishing wireless communication channel 50 between laptop computer 32 and WAP 52. As is known in the art, IEEE 802.11x specifications may use Ethernet protocol and carrier sense multiple access with collision avoidance (i.e., CSMA/CA) for path sharing. As is known in the art, Bluetooth is a telecommunications industry specification that allows e.g., mobile phones, computers, and personal digital assistants to be interconnected using a short-range wireless connection.

While the following discussion concerns medical imagery, this is for illustrative purposes only and is not intended to be a limitation of this disclosure, as other configurations are possible and are considered to be within the scope of this disclosure. For example, the following discussion may concern any type of clinical content (e.g., DNA sequences, EKG results, EEG results, blood panel results, lab results, etc.) and/or non-medical content.

Assume for the following example that users 36, 38 are medical service providers (e.g., radiologists) in two different medical facilities (e.g., hospitals, labs, diagnostic imaging centers, etc.). Accordingly and during the normal operation of these medical facilities, medical imagery may be generated by e.g., x-ray systems (not shown), MRI systems (not shown), CAT systems (not shown), PET systems (not shown) and ultrasound systems (not shown). For example, assume that user 36 generates medical imagery 54 and user 38 generates medical imagery 56; wherein medical imagery 54 may be stored locally on storage device 20 coupled to laptop computer 28 and medical imagery 56 may be stored locally on storage device 22 coupled to laptop computer 30. When locally storing medical imagery 54, 56, this medical imagery may be stored within e.g., a PACS (i.e., Picture Archiving and Communication System). Additionally/alternatively, the medical imagery (e.g., medical imagery 54, 56) may be stored on a cloud-based storage system (e.g., a cloud-based storage system (not shown) included within online platform 58).

Online platform process 10 may enable online platform 58 that may be configured to allow for the offering of various medical diagnostic services to users (e.g., users 36, 38) of online platform 58. For the following example, assume that user 40 is a medical research facility (e.g., the ABC Center) that performs cancer research. Assume that user 40 produced a process (e.g., automated analysis process 60) that analyzes medical imagery to identify anomalies that may be cancer. Examples of automated analysis process 60 may include but are not limited to an application or an algorithm that may process medical imagery (e.g., medical imagery 54 and medical imagery 56), wherein this application/algorithm may utilize artificial intelligence, machine learning and/or probabilistic modeling when analyzing the medical imagery (e.g., medical imagery 54 and medical imagery 56). Examples of such probabilistic modeling may include but are not limited to discriminative modeling (e.g., a probabilistic model for only the content of interest), generative modeling (e.g., a full probabilistic model of all content), or combinations thereof.

Further assume that user 42 is a medical research corporation (e.g., the XYZ Corporation) that produces applications/algorithms (e.g., automated analysis process 62) that analyze medical imagery to identify anomalies that may be cancer. Examples of automated analysis process 62 may include but are not limited to an application or an algorithm that may process medical imagery (e.g., medical imagery 54 and medical imagery 56), wherein this application/algorithm may utilize artificial intelligence, machine learning algorithms and/or probabilistic modeling when analyzing the medical imagery (e.g., medical imagery 54 and medical imagery 56). Examples of such probabilistic modeling may include but are not limited to discriminative modeling (e.g., a probabilistic model for only the content of interest), generative modeling (e.g., a full probabilistic model of all content), or combinations thereof.

Assume for the following example that user 40 (i.e., the ABC Center) wishes to offer automated analysis process 60 to others (e.g., users 36, 38) so that users 36, 38 may use automated analysis process 60 to process their medical imagery (e.g., medical imagery 54 and medical imagery 56, respectively). Further assume that user 42 (i.e., the XYZ Corporation) wishes to offer automated analysis process 62 to others (e.g., users 36, 38) so that users 36, 38 may use automated analysis process 62 to process their medical imagery (e.g., medical imagery 54 and medical imagery 56, respectively).

Accordingly, online platform process 10 and online platform 58 may allow user 40 (i.e., the ABC Center) and/or user 42 (i.e., the XYZ Corporation) to offer automated analysis process 60 and/or automated analysis process 62 (respectively) for use by e.g., user 36 and/or user 38. Therefore, online platform process 10 and online platform 58 may be configured to allow user 40 (i.e., the ABC Center) and/or user 42 (i.e., the XYZ Corporation) to upload a remote copy of automated analysis process 60 and/or automated analysis process 62 to online platform 58, resulting in automated analysis process 60 and/or automated analysis process 62 (respectively) being available for use via online platform 58. Accordingly, online platform process 10 may offer a plurality of computer-based medical diagnostic services (e.g., automated analysis process 60, 62) within the online platform (e.g., online platform 58), wherein online platform process 10 may identify the computer-based medical diagnostic services (e.g., automated analysis process 60, 62) that are available via online platform 58 and users (e.g., user 36, 38) may utilize these computer-based medical diagnostic services (e.g., automated analysis process 60, 62) to process the medical imagery (e.g., medical imagery 54 and medical imagery 56).

As could be expected, when users (e.g., user 36, 38) utilize these computer-based medical diagnostic services (e.g., automated analysis process 60, 62) to process the medical imagery (e.g., medical imagery 54 and medical imagery 56), it is foreseeable that unexpected results may occur. As discussed above, automated analysis processes 60, 62 may be utilized to identify anomalies within medical imagery (e.g., medical imagery 54 and medical imagery 56, respectively) that may be cancer. Unfortunately, misidentifications may occur. For example and once the medical imagery (e.g., medical imagery 54 and medical imagery 56) is processed by automated analysis processes 60, 62, the results of automated analysis processes 60, 62 may be reviewed by e.g., a radiologist. At this point, the radiologist(s) can determine if any misidentifications occurred. Examples of such misidentifications may include but are not limited to false negatives (e.g., when anomalies are present within medical imagery 54, 56 but automated analysis processes 60, 62 indicates that none exist) and false positives (e.g., when anomalies are not present within medical imagery 54, 56 but automated analysis processes 60, 62 indicates that some exist)

While the following discussion concerns the processing of medical imagery, this is for illustrative purposes only and is not intended to be a limitation of this disclosure, as other configurations are possible and are considered to be within the scope of this disclosure. For example, other types of medical information may be processed, such as DNA sequences, EKG results, EEG results, blood panel results, lab results, etc. Additionally, other types of information may be processed that need not be medical in nature. Accordingly and with respect to this disclosure, the content processed may be any type of content for which automated processing may be applicable, such as medical data, financial records, personal records, and identification information.

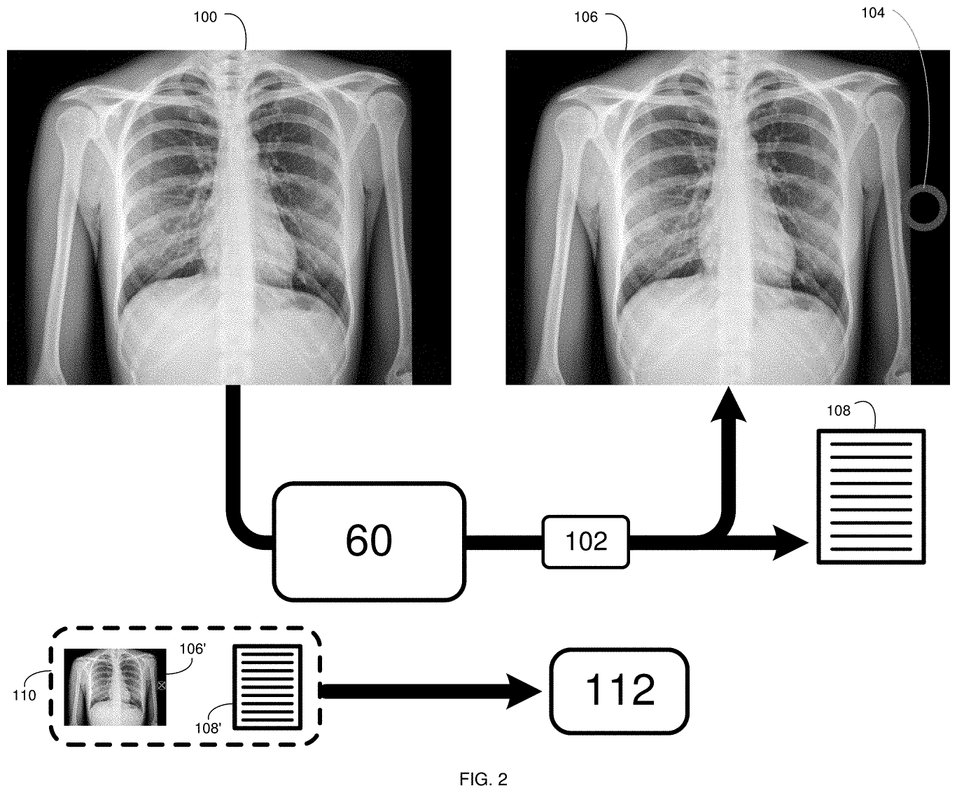

Referring also to FIG. 2 and for the following discussion, assume that user 38 (e.g., a radiologist) has a chest x-ray (e.g., chest x-ray 100) of a patient that is being processed by automated analysis process 60 to determine if there are any anomalies within chest x-ray 100. Assume for this example that automated analysis process 60 generates result set 102 that identifies one anomaly (e.g., anomaly 104). In this example, result set 102 may include annotated x-ray 106 and auto-populated report 108. For example, annotated x-ray 106 may visually locate anomaly 104; while auto-populated report 108 may be a radiologist report that is automatically generated by automated analysis process 60 and populated with the findings made by automated analysis process 60 (e.g., the identification, description and location of anomaly 104).

While result set 102 is shown to include annotated x-ray 106 and auto-populated report 108, this is for illustrative purposes only and is not intended to be a limitation of this disclosure, as other configurations are possible. As discussed above, while this example concerns medical imagery (e.g., chest x-ray 100), other types of data are possible and are considered to be within the scope of this disclosure (e.g., DNA sequences, EKG results, EEG results, blood panel results, lab results, non-medical information, etc. Accordingly and in such situations, result set 102 may be related to those other types of data that do not concern medical imagery and/or are not medical in nature.

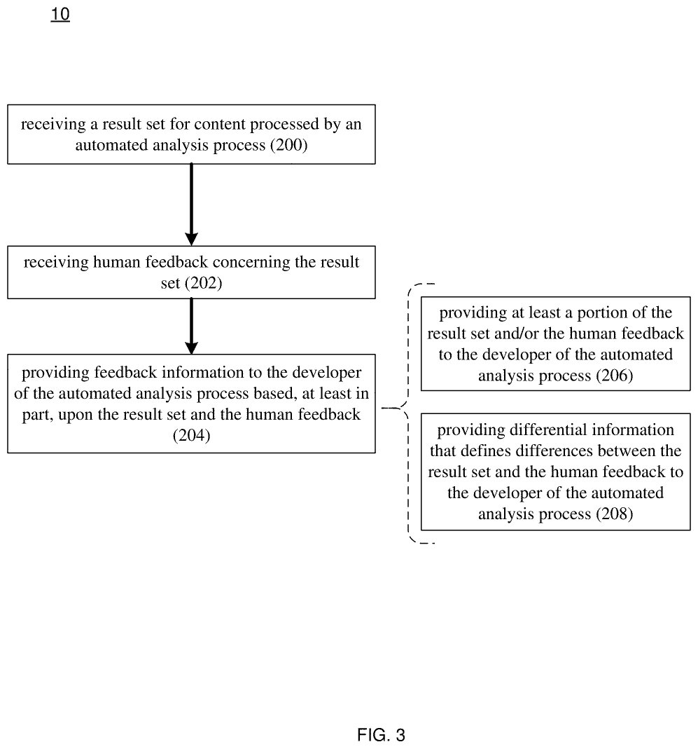

Referring also to FIG. 3, online platform process 10 may receive 200 a result set (e.g., result set 102) for content (e.g., chest x-ray 100) processed by an automated analysis process (e.g., automated analysis process 60), which may be reviewed by user 38 (e.g., a radiologist). Assume that upon user 38 (e.g., a radiologist) who is reviewing result set 102 determines that result set 102 is inaccurate, as e.g., chest x-ray 100 is clean (i.e., it does not show any anomalies) and the identified anomaly (i.e., anomaly 104) is shown to be located outside of the body.

Accordingly, user 38 (e.g., a radiologist) may revise result set 102 to generate human feedback 110 concerning result set 102. Human feedback 110 may generally concern the accuracy of result set 102. As discussed above and in this example, result 102 is inaccurate, as it contains a false positive (i.e., falsely identifies anomaly 104). Accordingly, human feedback 110 may identify/document such inaccuracies within result set 102 and/or may include amendments to result set 102.

For example, human feedback 110 may include amendments to annotated x-ray 106 (resulting in amended x-ray 106′) and/or amendments to auto-populated report 108 (resulting in amended report 108′). For example, amended x-ray 106′ may be a revised/annotated version of annotated x-ray 106 that (in this particular example) removes any indication of anomaly 104). Additionally, amended report 108′ may be a revised/annotated version of auto-populated report 108 that (in this particular example) removes any reference of anomaly 104).

Online platform process 10 may receive 202 human feedback 110 concerning result set 102 and may provide 204 feedback information (e.g., feedback information 112) to the developer (e.g., user 40 of the ABC Center) of automated analysis process 60 based, at least in part, upon result set 102 and human feedback 110.

When providing 204 feedback information (e.g., feedback information 112) to the developer (e.g., user 40 of the ABC Center) of automated analysis process 60, online platform process 10 may provide 206 at least a portion of result set 102 and/or human feedback 110 to the developer (e.g., user 40 of the ABC Center) of automated analysis process 60. For example, feedback information 112 may include all or a portion of annotated x-ray 106 (which shows anomaly 104) and all or a portion of amended x-ray 106′ (which deletes anomaly 104) to illustrate any inaccuracies associated with automated analysis process 60. Further, feedback information 112 may include all or a portion of auto-populated report 108 (which discusses anomaly 104) and all or a portion of amended report 108′ (which deletes reference to anomaly 104) to illustrate any inaccuracies associated with automated analysis process 60.

Accordingly and when providing 204 feedback information (e.g., feedback information 112) to the developer (e.g., user 40 of the ABC Center) of automated analysis process 60, online platform process 10 may provide 208 differential information that defines differences between result set 102 and human feedback 110 to the developer (e.g., user 40 of the ABC Center) of automated analysis process 60. Specifically and in this example, feedback information 112 may identify that automated analysis process 60 defined anomaly 104 within result set 102, while human feedback 110 did not define such an anomaly, thus indicating that automated analysis process 60 generated a false positive.

While the above discussion concerns automated analysis process 60 producing inaccurate results and there being a differential between result set 102 and human feedback 110, it is understood that there will be little (if any) differential between result set 102 and human feedback 110 if automated analysis process 60 produced accurate results.

The developer (e.g., user 40 of the ABC Center) of automated analysis process 60 may utilize feedback information 112 to gauge the quality/accuracy of automated analysis process 60 and troubleshoot any problems identified therein. For example and through the use of feedback information 112, the source of any misidentifications (e.g., false negatives or false positives) may be identified, as feedback information 112 may include e.g., a description of the problem (e.g., anomaly 104 being shown to be located outside of the body), the problematic result set (e.g., result set 102), and the input image (e.g., chest x-ray 100).

Unfortunately, the above-described procedures may get complicated when dealing with confidential data (such as medical imagery), as various laws, rules and regulations (e.g., HIPAA Privacy Rules) strictly control the dissemination of confidential medical data. For example, the HIPAA Privacy Rules establishes national standards to protect individuals' medical records and other personal health information and applies to health plans, health care clearinghouses, and those health care providers that conduct certain health care transactions electronically. Additionally, it is good practice not to share such confidential data even if permitted by law, rule and regulation. Accordingly, online platform process 10 may be configured to allow for the submission of such feedback information 112 without the submission of such confidential data. Therefore, feedback information 112 may be processed to remove confidential data (generally) and in accordance with one or more medical data privacy rules (specifically), resulting in the generation of non-confidential data that is related to the confidential data (e.g., chest x-ray 100).

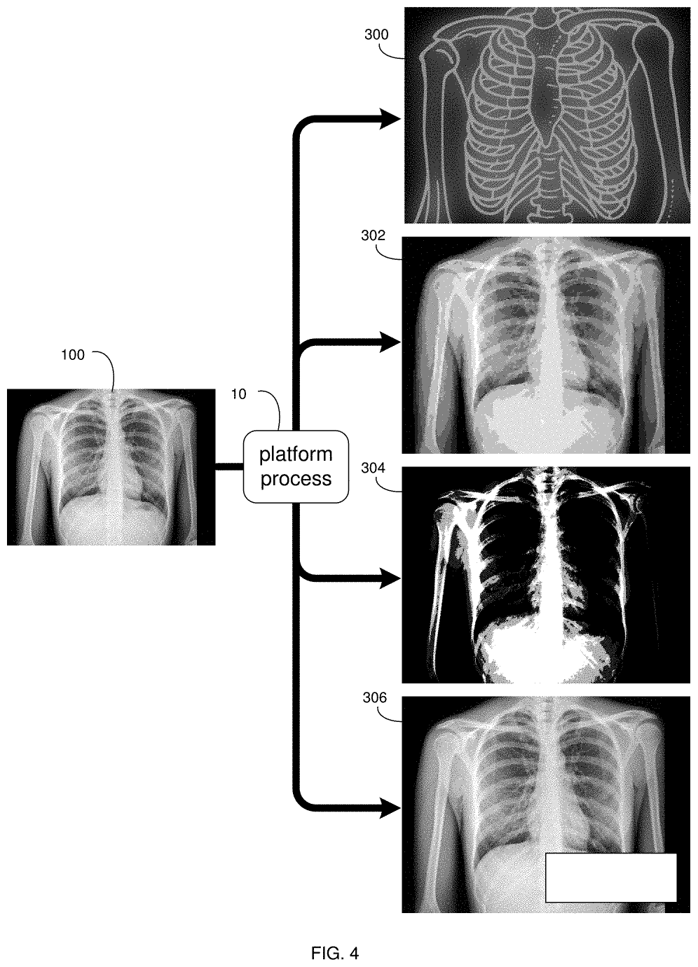

Referring also to FIG. 4, online platform process 10 may process chest x-ray 100 to generate (in this example) one or more instantiations of non-confidential data (e.g., non-confidential data 300, non-confidential data 302, non-confidential data 304, and non-confidential data 306), wherein each of these instantiations is related to the confidential data (e.g., chest x-ray 100). When processing the confidential data (e.g., chest x-ray 100) to generate these instantiations of non-confidential data (e.g., non-confidential data 300, non-confidential data 302, non-confidential data 304, and non-confidential data 306) that is related to the confidential data (e.g., chest x-ray 100), online platform process 10 may apply one or more medical data privacy rules (e.g., HIPAA Rules) to the confidential data (e.g., chest x-ray 100) to generate the non-confidential data (e.g., non-confidential data 300, non-confidential data 302, non-confidential data 304, and non-confidential data 306) that is related to the confidential data (e.g., chest x-ray 100).

For example and after applying these medical data privacy rules (e.g., HIPAA Rules) to the confidential data (e.g., chest x-ray 100), the non-confidential data (e.g., non-confidential data 300, non-confidential data 302, non-confidential data 304, and non-confidential data 306) may include one or more of:
instantiations of obscured data, wherein online platform process 10 may obscure one or more portions of the confidential data (e.g., chest x-ray 100) to generate non-confidential data.
instantiations of pixelated data, wherein online platform process 10 may pixelate one or more portions of the confidential data (e.g., chest x-ray 100) to generate non-confidential data.
instantiations of ambigutized data, wherein online platform process 10 may ambigutize one or more portions of the confidential data (e.g., chest x-ray 100) to generate non-confidential data.
instantiations of redacted data, wherein online platform process 10 may redact one or more portions of the confidential data (e.g., chest x-ray 100) to generate non-confidential data.

Accordingly and by obscuring/pixelating/ambigutizing/redacting some or all of the confidential data (e.g., chest x-ray 100), the newly-generated non-confidential data may adhere to and meet the requires of the medial data privacy rules (e.g., the HIPAA rules).

### GENERAL

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
receiving a result set for content processed by an automated analysis process;
receiving human feedback concerning the result set; and
providing feedback information to the developer of the automated analysis process based, at least in part, upon the result set and the human feedback.

2. The computer-implemented method of claim 1 wherein the result set is an auto-populated report.

3. The computer-implemented method of claim 2 wherein the human feedback includes amendments to the auto-populated report.

4. The computer-implemented method of claim 1 wherein the human feedback concerns the accuracy of the result set.

5. The computer-implemented method of claim 1 wherein the human feedback includes amendments to the result set.

6. The computer-implemented method of claim 1 wherein the content is medical imagery.

7. The computer-implemented method of claim 1 wherein providing feedback information to the developer of the automated analysis process includes:
providing at least a portion of the result set and/or the human feedback to the developer of the automated analysis process.

8. The computer-implemented method of claim 1 wherein providing feedback information to the developer of the automated analysis process includes:
providing differential information that defines differences between the result set and the human feedback to the developer of the automated analysis process.

9. The computer-implemented method of claim 1 wherein the feedback information is processed to remove confidential data.

10. The computer-implemented method of claim 1 wherein the feedback information is processed to remove confidential data in accordance with one or more medical data privacy rules.

11. A computer program product residing on a computer readable medium having a plurality of instructions stored thereon which, when executed by a processor, cause the processor to perform operations comprising:
receiving a result set for content processed by an automated analysis process;
receiving human feedback concerning the result set; and
providing feedback information to the developer of the automated analysis process based, at least in part, upon the result set and the human feedback.

12. The computer program product of claim 11 wherein the result set is an auto-populated report.

13. The computer program product of claim 12 wherein the human feedback includes amendments to the auto-populated report.

14. The computer program product of claim 11 wherein the human feedback concerns the accuracy of the result set.

15. The computer program product of claim 11 wherein the human feedback includes amendments to the result set.

16. The computer program product of claim 11 wherein the content is medical imagery.

17. The computer program product of claim 11 wherein providing feedback information to the developer of the automated analysis process includes:
providing at least a portion of the result set and/or the human feedback to the developer of the automated analysis process.

18. The computer program product of claim 11 wherein providing feedback information to the developer of the automated analysis process includes:
providing differential information that defines differences between the result set and the human feedback to the developer of the automated analysis process.

19. The computer program product of claim 11 wherein the feedback information is processed to remove confidential data.

20. The computer program product of claim 11 wherein the feedback information is processed to remove confidential data in accordance with one or more medical data privacy rules.

21. A computing system including a processor and memory configured to perform operations comprising:
receiving a result set for content processed by an automated analysis process;
receiving human feedback concerning the result set; and
providing feedback information to the developer of the automated analysis process based, at least in part, upon the result set and the human feedback.

22. The computing system of claim 21 wherein the result set is an auto-populated report.

23. The computing system of claim 22 wherein the human feedback includes amendments to the auto-populated report.

24. The computing system of claim 21 wherein the human feedback concerns the accuracy of the result set.

25. The computing system of claim 21 wherein the human feedback includes amendments to the result set.

26. The computing system of claim 21 wherein the content is medical imagery.

27. The computing system of claim 21 wherein providing feedback information to the developer of the automated analysis process includes:
providing at least a portion of the result set and/or the human feedback to the developer of the automated analysis process.

28. The computing system of claim 21 wherein providing feedback information to the developer of the automated analysis process includes:
providing differential information that defines differences between the result set and the human feedback to the developer of the automated analysis process.

29. The computing system of claim 21 wherein the feedback information is processed to remove confidential data.

30. The computing system of claim 21 wherein the feedback information is processed to remove confidential data in accordance with one or more medical data privacy rules.
